(19) United States
(12) Patent Application Publication
(10) Pub. No.: US 2025/0195858 A1
(43) Pub. Date: Jun. 19, 2025

(54) ACCESS GRAFT SYSTEMS AND METHODS

(71) Applicant: W. L. Gore & Associates, Inc., Newark, DE (US)

(72) Inventors: Edward H. Cully, Clarksville, VA (US); Paul J. Fordenbacher, Scottsdale, AZ (US); Rachel Radspinner, Flagstaff, AZ (US)

(21) Appl. No.: 18/847,383

(22) PCT Filed: Mar. 16, 2023
(86) PCT No.: PCT/US2023/015352

§ 371 Date: Sep. 16, 2024

Related U.S. Application Data
(60) Provisional application No. 63/320,386, filed on Mar. 16, 2022.

(51) Int. Cl. A61M 39/02; A61M 1/36

(52) U.S. Cl. CPC A61M 39/0208; A61M 1/3655; A61M 2039/0238; A61M 2205/0216; A61M 2205/0266; A61M 2205/0272

(57) ABSTRACT

A device for supporting an elongate tubular member, the device including a support member having an interior surface defining a receiving cavity, the support member further defining a circumferential opening, wherein the receiving cavity is operable to receive an elongate tubular member therein, the support member operable to place the elongate tubular member into a state of compression about the circumference.

### CROSS-REFERENCE TO RELATED APPLICATION

This application is a national phase application of PCT Application No. PCT/US2023/015352, internationally filed on Mar. 16, 2023, which claims the benefit of Provisional Application No. 63/320,386, filed Mar. 16, 2022, which are incorporated herein by reference in their entireties for all purposes.

### FIELD

The present disclosure relates generally to implantable conduits. More specifically, the present disclosure relates to apparatuses, systems, and methods for providing a self-sealing graft, including an implantable graft.

### BACKGROUND

Vascular access grafts are used for patients with conditions that need regular intraluminal access. For example, patients undergoing dialysis treatment require regular cannulation of the graft with a dialysis needle inserted through the graft wall into the graft lumen which is coupled to the system of blood lines that allow blood to be circulated between the patient and the dialysis machine. Although vascular access grafts are operable to be self-sealing, repeated entry can break down the graft leading to blood leaking out of the graft.

Another difficulty with repeated access can be the process of gaining access to the lumen of the graft by the needle. Often, the graft may present a relatively small target or surface area through which a medical professional or trained patient from self-administered treatment must insert a needle to gain access to the graft. Failure to gain access results in the need to reinsert the needle which causes further discomfort to the patient.

Another difficulty arises when the needle is advanced too far and extends completely through the access graft. It is relatively difficult to determine the depth of the access graft and therefore it is difficult to discern the appropriate depth to which a needle should be inserted to be in the lumen of the access graft.

There is current and ongoing development of at-home self-administered dialysis. At home and overnight dialysis has the capacity to provide significant quality of life improvements for those on dialysis, however, safe and usable equipment is necessary to provide the patient the ability to safely self-administer the treatments.

What is needed are improvements in providing and/or supporting tubular structures for medical procedures.

### SUMMARY

According to one example (“Example 1”), a device for supporting an elongate tubular member, the device includes a support member having an interior surface defining a receiving cavity, the support member further defining a circumferential opening, wherein the receiving cavity is operable to receive an elongate tubular member therein, the support member operable to place the elongate tubular member into a state of compression about the circumference.

According to another example (“Example 2”), further to Example 1, the receiving cavity is adhesively coupled to the elongate tubular member.

According to another example (“Example 3”), further to Example 1 or 2, the support member comprises a material resistant to puncture by a needle at typical forces exerted manually by a user.

According to another example (“Example 4”), further to Example 3, the support member comprises a bioabsorbable material.

According to another example (“Example 5”), further to any one of Examples 1-4, the circumferential opening of the support member is from about 10% to about 90% of a circumference of elongate tubular member.

According to another example (“Example 6”), further to any one of Examples 1-4, the interior surface of the support member presents an oval cross-section.

According to another example (“Example 7”), further to any one of Examples 1-4, the interior surface of the support member presents a circular cross-section.

According to another example (“Example 8”), further to any one of Examples 6-7, the support member is operable to retain the same cross-sectional area of the elongate tubular member within the receiving cavity and external of the receiving cavity.

According to another example (“Example 9”), further to any one of Examples 1-8, the support member has two opposing arms defining the circumferential opening, the two opposing arms being interconnected by a body and including a material having a durometer that is lower than a durometer of the body.

According to another example (“Example 10”), further to any one of Examples 8-9, the two opposing arms impart a compressive force to the elongate tubular member.

According to another example (“Example 11”), further to any one of Examples 9-10, the two opposing arms portions are formed from a shape memory material.

According to another example (“Example 12”), further to Example 11, the shape memory material is nitinol.

According to another example (“Example 13”), further to any one of Examples 9-12, the body is thicker than the two opposing arms.

According to another example (“Example 14”), further to any one of Examples 1-13, the support member includes a plurality of segments along a longitudinal length operable to facilitate flexion of the elongate tubular member when positioned with the support member.

According to another example (“Example 15”), further to Example 14, the plurality of segments are discrete elements.

According to another example (“Example 16”), further to Example 14, the plurality of segments are an integral member forming the support member, the support member including an extension portion extending between adjacent segments, the extension portion including a thinned profile operable to facilitate flexion.

According to another example (“Example 17”), further to any one of Examples 14-16, the plurality of segments facilitates flexion of the elongate tubular member.

According to another example (“Example 18”), the support member is resistant to puncture.

According to another example (“Example 19”), further to any one of Examples 1-17, wherein the support member is operable to place the tubular member into the state of compression.

According to another example (“Example 20”), further to any of Examples 1-5, further includes a location device operable to magnetically coupled to the support member, the location device forming a donut hole through which a needle may be inserted.

According to another example (“Example 21”), further to Example 20, the location device includes indicators about a periphery of the donut hole indicating different positions for needle insertion.

According to another example (“Example 22”), a method of assembling the support member of any one of Example 1-7, onto the elongate tubular member comprises overlapping the support member and the elongate tubular member to impart compressive force to the tubular member, wherein a diameter of the support member in the overwrap is less than a diameter of the support member prior to applying the overwrap.

According to another example, (“Example 23”), overwrapping is done after applying an adhesive coupling to the support member and the elongate tubular member and curing the adhesive.

According to another example (“Example 24”), a method of assembling the support member of any one of Examples 1-7 onto the elongate tubular member comprises elongating the tubular member so as to neck down an outer diameter of the elongate tubular member to be received within the receiving cavity of the support member, wherein the receiving cavity of the support member has a smaller diameter than the outer diameter of the tubular member when the tubular member is not in a state of compression.

According to another example (“Example 25”), a method of assembling the support member of Example 24 onto the elastomeric tubular member, comprising deflecting the arms away from the body enlarging the circumferential opening operable for the tubular member to be received in the receiving cavity.

According to another example (“Example 26”), an AV access graft, includes an elongate tubular member defining a lumen and an elongate support member defining a receiving cavity and defining a circumferential opening, wherein the receiving cavity is operable to receive the elongate tubular member therein, the elongate support member operable to place the tubular member into a state of circumferential compression.

According to another example (“Example 27”), further to Example 26, the receiving cavity is operable to receive an adhesive to couple the elongate tubular member within the receiving cavity.

According to another example (“Example 28”), further to any one of the Examples 25-26, the elongate tubular member is an artery or vein.

According to another example (“Example 29”), further to any one of Examples 25-26, the elongate tubular member is a biological material, synthetic material, or a combination thereof.

According to another example (“Example 30”), further to any one of Examples 25-28, the elongate tubular member in the state of circumferential compression is operable to recover and seal after being punctured by a 14-gauge, 15-gauge, 16-gauge, or a 17-gauge needle.

According to another example (“Example 31”), further to any one of Examples 25-28, an outer dimension of the elongate tubular member when in the state of circumferential compression is in a range from about 1% to about 10% relative to the outer dimension of the elongate tubular member when not in a state of circumferential compression.

According to another example (“Example 32”), further to any one of Examples 25-28, the elongate tubular member includes an oval-shaped cross section along at least a portion of the elongate tubular member where the elongate tubular member is supported by the support member.

According to another example (“Example 33”), further to any one of Examples 25-31, the support member is an elongate C-shaped clip.

According to another example (“Example 34”), an AV access graft includes an elastomeric, elongate tubular member including a first longitudinal portion having an oval-shaped cross section and resists deformation along the first longitudinal portion such that the oval-shaped cross section at the first longitudinal portion is retained when exposed to biological forces when implanted in a body.

According to another example (“Example 35”), further to Example 34, the elastomeric, elongate tubular member includes a rigidified layer at the first longitudinal portion.

According to another example (“Example 36”), further to Example 34, the elastomeric, elongate tubular member includes a shape memory layer at the first longitudinal portion.

According to another example (“Example 37”), further to Example 34, the elastomeric, elongate tubular member is treated to include the oval-shaped cross section at the first longitudinal portion.

According to another example (“Example 38”), further to Example 37, the elastomeric, elongate tubular member is heat set to include the oval-shaped cross section at the first longitudinal portion.

According to another example (“Example 39”), further to Example 34, the elastomeric, elongate tubular member includes an integral brace member at the first longitudinal portion, the brace member imparting the oval-shaped cross section.

According to another example (“Example 40”), further to Examples 34-39, the elastomeric, elongate tubular member at the first longitudinal portion includes a first elongate portion and a second elongate portion defining the oval-cross section, wherein the first elongate portion is operable to self-seal after a puncture.

According to another example (“Example 41”), further to Examples 34-39, the second elongate portion is reinforced to resist puncture.

According to another example (“Example 42”), an AV access graft includes an elongate tubular member including a first longitudinal portion having an integral support member over a portion of a circumference of the elastomeric, elongate tubular member at the first longitudinal portion.

According to another example (“Example 43”), further to Example 42, the integral support member includes a higher durometer than a remaining portion of the circumference of the elongate tubular member at the first longitudinal portion.

According to another example (“Example 44”), further to any one of Examples 42-43, the elongate tubular member is a fluoropolymer.

According to another example (“Example 45”), further to any one of Examples 42-44, the elongate tubular member includes a circular profile.

According to another example (“Example 46”), further to any one of Examples 42-45, the elongate tubular member includes an oval profile.

According to another example (“Example 47”), further to Example 46, the integral support member at least partially imparts the oval profile onto the elongate tubular member.

According to another example (“Example 48”), further to any one of Examples 42-47, the elongate tubular member is self-sealing.

According to another example (“Example 49”), further to any one of Examples 42-48, the support member is adhered or bonded to the elongate tubular member.

According to another example (“Example 50”), further to any one of Examples 42-49, the elongate tubular member is formed of a single layer of material.

According to another example (“Example 51”), further to Example 50, the support member is a densified region of the elongate tubular member.

According to another example (“Example 52”), further to any one of Examples 42-50, the support member is positioned at about 25% of a circumference of the elongate tubular member at a first longitudinal position.

According to another example (“Example 53”), further to any one of Examples 42-51, the elongate tubular member is free from the support member at each end of the elastomeric, elongate tubular member.

According to another example (“Example 54”), further to any one of Examples 42-53, the integral support member is a rigidified layer.

According to another example (“Example 55”), further to any one of Examples 42-54, the integral support member is a tubular member and supports a majority of a length of the elongate tubular member.

According to another example (“Example 56”), a device for supporting a tubular member, the device comprising a segmented support member including a plurality of longitudinal segments, the segmented support member having an interior surface defining a receiving cavity, wherein the receiving cavity is operable engage the tubular member therein, each longitudinal segment in the plurality of longitudinal segments at least partially surrounding a portion of the tubular member.

According to another example (“Example 57”), further to Examples 56, wherein each longitudinal segment of the plurality of longitudinal segments are discrete elements and are spaced apart from each other.

According to another example (“Example 58”), further to Example 57, wherein the segmented support member is substantially rigid, and the tubular member is able to bend due to the longitudinal segments being spaced apart.

According to another example (“Example 59”), further to Example 58, wherein a distance between each longitudinal segment in the plurality of longitudinal segments is determines the degree of bending of the tubular member.

According to another example (“Example 60”), further to Example 58, wherein each longitudinal segment in the plurality of longitudinal segments contacts an adjacent longitudinal segment along an inside curvature when the tubular member is bent.

According to another example (“Example 61”), further to any one of Examples 56-60, the tubular member is adhered or bonded within the receiving cavity.

According to another example (“Example 62”), further to any one of Examples 56-61, the tubular member flexes along a length of the tubular member when engaged with the segmented support member.

According to another example (“Example 63”), further to any one of Examples 56-62, wherein the segmented support segment is resistant to puncture.

According to another example (“Example 64”), further to any one of Examples 56-63, wherein each longitudinal segment in the plurality of longitudinal segments are connected together by an extension portion.

According to another example (“Example 65”), further to Example 64, wherein the extension portion is integral to the segmented support member.

According to another example (“Example 66”), further to Example 64, wherein the extension portion is coupled to or adhered to the segmented support member.

According to another example (“Example 67”), further to any one of Examples 64-66, wherein the extension portion positions each longitudinal segments in the plurality of longitudinal segments at a fixed distance from adjacent longitudinal segments when in a neutral orientation.

According to another example (“Example 68”), further to any one of Examples 64-67, wherein the extension portion is operable to bend or deflect to facilitate a curvature of the tubular member.

According to another example (“Example 69”), further to any one of Examples 64-68, wherein the extension portion is made of a shape memory material including nitinol.

According to another example (“Example 70”), further to any one of Examples 64-69, wherein the extension portion has a thinned profile to facilitate flexion of the tubular member.

According to another example (“Example 71”), further to any one of Examples 64-70, wherein the segmented support member is operable to place the tubular member in a state of longitudinal compression.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of a device including a tubular member being supported by a support member, in accordance with an embodiment;

FIG. 2 is an end view of a support member with an oval profile, in accordance with an embodiment;

FIG. 3 is a perspective view of a support member with an oval profile, in accordance with an embodiment;

FIG. 4 is an end view of a support member with a circular profile, in accordance with an embodiment;

FIG. 5 is an end view of a support member with a body that is thicker relative to the arms of the support member, in accordance with an embodiment;

FIG. 6 is an end view of needle access to an interior lumen of a tubular member supported by a support member, in accordance with an embodiment;

FIG. 7 is a top view of needle access to an interior lumen of a tubular member supported by a segmented support member, in accordance with an embodiment;

FIG. 8 is a perspective view of a tubular member supported by a segmented support member, in accordance with an embodiment;

FIG. 9 is a top view of a support member being supported by a segmented support member, the tubular member including a bend, in accordance with an embodiment;

FIG. 10A is a side view of a support member including segments coupled together by an extension portion, in accordance with an embodiment;

FIG. 10B is a perspective view of a support member including segments coupled together by an extension portion, in accordance with an embodiment;

FIG. 10C is a bottom view of a support member including segments including a chevron-shaped configuration, in accordance with an embodiment;

FIG. 11 is a perspective view of a tubular member including an integral support member, in accordance with an embodiment;

FIG. 12 is a section view of a tubular member having an integral support member, in accordance with an embodiment;

FIG. 13 is a section view of a tubular member having an integral support member positioned on an exterior surface of the tubular member, in accordance with an embodiment;

FIG. 14 is a section view of a tubular member having an integral support member positioned on an interior surface of the tubular member, in accordance with an embodiment;

FIG. 15 is a perspective view of a tubular member having an integral support member positioned at a first longitudinal position, the tubular member being free of the support member along at least a portion of the tubular member, in accordance with an embodiment;

FIG. 16 is a section view of a tubular member having an integral support member that is positioned at less than 50% of the circumference of the tubular member, in accordance with an embodiment;

FIGS. 17A and 17B are sectional views of a device being overwrapped, in accordance with an embodiment;

FIG. 18 is an exemplary method of manufacturing a device, in accordance with an embodiment; and

FIG. 19 is view of a location device for helping locate an implantable device when the implantable device is implanted in a patient, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

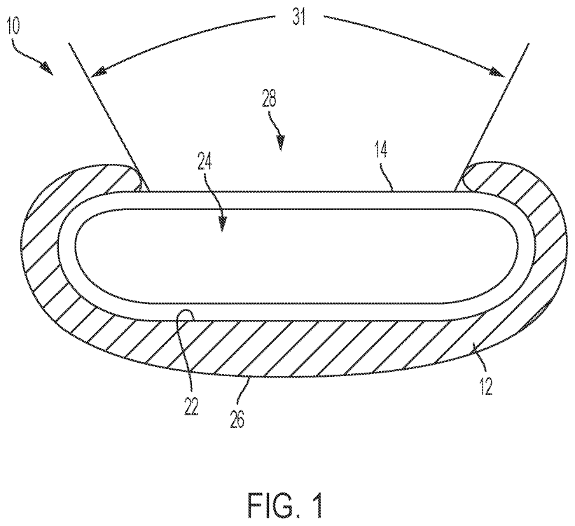

The device shown in FIG. 1 is provided as an example of the various features of the one embodiment of the device and, although the combination of those illustrated features is clearly within the scope of invention, that example and its illustration is not meant to suggest the inventive concepts provided herein are limited from fewer features, additional features, or alternative features to one or more of those features shown in FIG. 1.

Referring to FIG. 1, a device 10 is provided for repeated and repeatable intralumenal access. For example, intravenous access may be necessary for such procedures as dialysis that requires repeated access at an access site. The device 10 facilitates access by providing support of the access site to limit leakage after a single puncture or multiple punctures and, in some embodiments, an increased surface area which may be implemented for providing intralumenal access.

In some embodiments, the device 10 includes a support member 12 and a tubular member 14. The support member 12 is operable to support the tubular member 14 in a specific configuration and/or in a compressed state to increase the surface area of a target site and/or to limit the leakage of the tubular member 14 during and after puncture. The support member 12 may be separate from the tubular member 14 such that it is installed on the tubular member 14 (e.g., removeable, attached after manufacture, etc.) or may be provided integral with the tubular member 14 (formed together with, inseparably secured to, etc.). Each embodiment (i.e., separate and integrated support member 12 and tubular member 14) is discussed in more detail herein.

Referring still to FIG. 1, the embodiment illustrated demonstrates a support member 12 that partially circumferentially surrounds a tubular member 14 and provides structural support the tubular member 14. The tubular member 14 may take a variety of forms, including grafts, synthetic vessels, native vessels, and so forth. The support member 12 can be used to support the tubular member 14 for various purposes, including but not limited to arterio-venous access (“AV access”), for example repeated access such as for dialysis. Although the device 10 is discussed in detail with respect to AV access, the device 10 is contemplated and discussed with respect to other purposes.

In some embodiments including the embodiment shown in FIG. 1, the support member 12 is an elongate C-shaped clip (see also FIG. 3 illustrating the elongate nature of the support member 12). The support member 12 has an interior surface 22 defining a receiving cavity 24 and an exterior surface 26. The interior surface 22 includes a concave profile and the exterior surface includes a convex profile. As illustrated, the interior and exterior surfaces 22 are curved to have the concave and convex profiles; however, it is within the scope of the disclosure that the interior and exterior surfaces are not curved, or are not entirely curved (e.g., instead being formed of straight segments) that generally provide for concave and convex profiles. The receiving cavity 24 is operable to receive the tubular member 14 such that the tubular member 14 is positioned and retained within the receiving cavity 24 and positioned against the interior surface 22 of the support member 12.

Referring to FIG. 3, the support member 12 includes a circumferential opening 28 which provides access to the receiving cavity 24 of the support member 12. In some embodiments, the circumferential opening 28 extends along the entire longitudinal length of the support member 12 such that the tubular member 14 may be advanced into the receiving cavity 24 by passing the tubular member 14 through the circumferential opening 28. This allows the tubular member 14 to remain intact during installation with the support member 12. This can be important when the tubular member 14 is a native vessel or an implanted graft, thus eliminating the need to thread an end of the tubular member 14 through the support member 12, which would require bisecting the tubular member 14 in order to thread an end of the tubular member 14 through the support member 12.

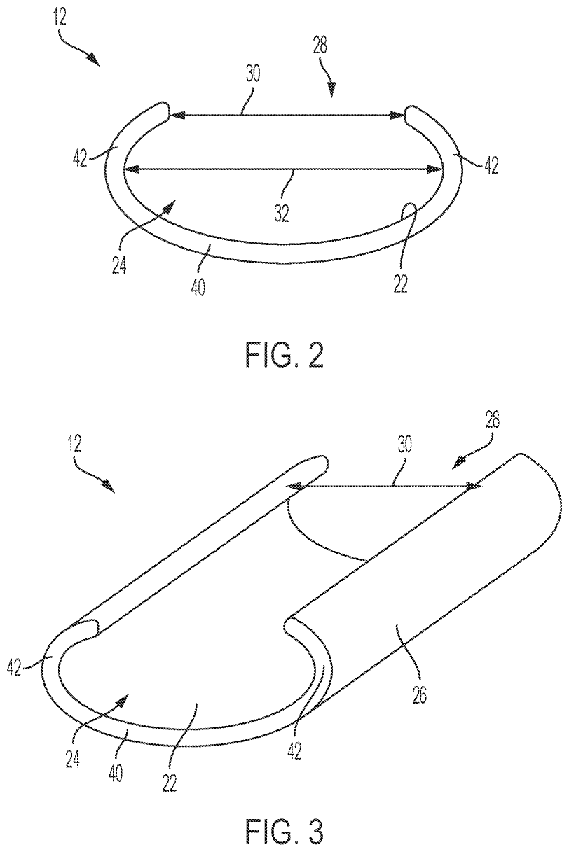

The width 30 of the circumferential opening 28 of the support member 12 is less than an inner dimension 32 of the receiving cavity 24 (e.g., the widest portion of the inner diameter for non-circular profiles, where represents the distance between opposing sides of the inner surface, see FIG. 2). This allows the support member 12 to surround the tubular member 14 more than 180 degrees and less than 360 degrees when the tubular member 14 is positioned in the receiving cavity 24. For example, when the tubular member 14 is installed in the support member 12, the support member 12 may surround the tubular member 14 from about 190 degrees to about 215 degrees, from about 215 degrees to about 240 degrees, from about 240 degrees to about 265 degrees, from about 265 degrees to about 290 degrees, and from about 290 degrees to about 325 degrees. The width 30 of the circumferential opening 28 may be larger in some embodiments and smaller in other embodiments. The gripping force of the support member 12 (as well as the compression force which is described hereafter) may be increased when the width 30 of the circumferential opening 28 is relatively less than in embodiments in which the width 30 of the circumferential opening 28 is greater. For example, the gripping force may be increased by increasing the width of the circumferential opening 28 and decreased by increasing the width of the circumferential opening 28.

In some embodiments, the support member 12 is sized such that an inner circumference of the support member 12 (including the width 30 of the circumferential opening 28) is smaller than an outer circumference of the tubular member 14. By sizing the inner circumference of the support member 12 to be smaller than the outer circumference of the tubular member 14, the tubular member is placed into a state of compression (e.g., compression about the circumference of the tubular member 14) when the tubular member 14 is engaged with the support member 12. The compression reduces leakage and facilitates resealing of the tubular member 14 during and after puncture (e.g., a needle puncture for dialysis). The compression pushes the sides of the opening formed by the puncture back in contact with each other to help prevent or reduce leakage of fluid through the puncture. The amount of compression can be altered by adjusting the relative circumferences of the support member 12 and the tubular member 14. Greater compression may be achieved by providing a narrower width 30 of the circumferential opening 28, thus placing the tubular member 14 in increased contact with the interior surface 22 of the support member 12 (for example, the compression may be increased by reducing the width of the circumferential opening 28). A narrower width 30 of the circumferential opening 28 may also limit bulging of the tubular member 14 out of the support member 12 at the circumferential opening 28, which can reduce the compression of the tubular member 14 including at the portion bulging outward from the circumferential opening 28. Thus, the narrower width 30 may facilitate use of tubular members 14 with increased outer diameters to facilitate increased compression of the tubular member 14.

In some embodiments, the circumferential opening 28 of the elongate support member 12 is from about 10% to about 90% of the circumference of the support member 12. In some embodiments, the circumferential opening 28 of the elongate support member 12 is from about 25% to about 75% of the circumference of the support member 12. In embodiments with the circumferential opening 28 being greater than 50% of the circumference of the support member 12, the compression of the elongate support member 12 may be a localized compression. For example, the elongate support member 12 may include pores (e.g., microscopic pores) that are compressed but the elongate tubular member 14 is not compressed about a full circumference. In embodiments with the circumferential opening 28 being less than 50% of the circumference of the support member 12, the compression of the elongate support member 12 may be a generalized compression. For example, the elongate support member 12 may be compressed about a full circumference. In some embodiments the width 30 of the circumferential opening 28 defines the cannulation area of the device 12 (e.g., in those embodiments used for cannulation such as hemodialysis). This may also define the cannulation angle 31 of the device 10 which is the angle at which cannulation access to the tubular member 14 may be achieved (see FIG. 1).

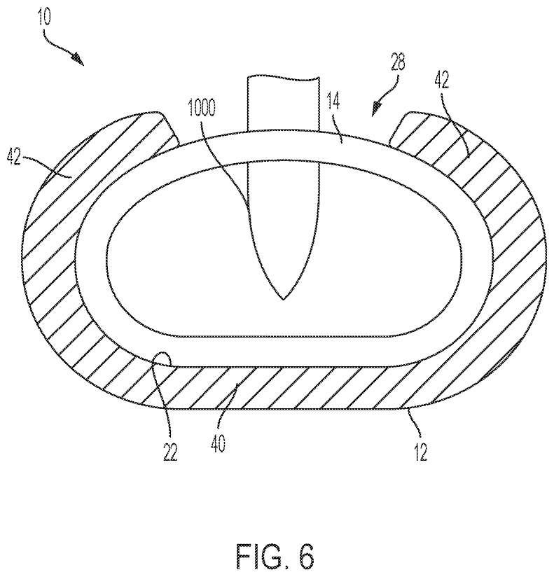

In some embodiments, the support member 12 comprises a material substantially resistant to puncture by a needle under typical forces exerted manually by a user. This limits the travel of the needle through the device 10. For example, the tubular member 14 may be punctured for intralumenal access at the circumferential opening 28 of the support member 12 but is limited through travelling through the opposite side of the tubular member because the body of the support member 12 is not penetrated by the needle. This limits the ability of a user to potentially extend a needle completely through the tubular member 14, which can result in excessive bleeding, delivery of fluids outside of the lumen of the tubular member 14, ineffective uptake of fluids from the lumen of the tubular member 14, and so forth. One embodiment demonstrating a needle 1000 that is restricted from travelling through the support member 12 is illustrated in FIG. 6. The support member 12 comprises a material resistant to puncture by a needle (e.g., nitinol). It is noted that the needle 1000 may puncture a portion of the back side of the tubular member 14, but is restricted from moving beyond by the support member 12. Thus, the lumen or bore of the needle 1000 is in fluid communication with the lumen of the tubular member 14 and fluids can be delivered to and/or received by the lumen of the tubular member 14. Various materials may be implemented in constructing the support member 12 to be resistant to needle puncture, including but not limited to biocompatible metals, alloys, polymers, and so forth.

In some embodiments, to improve cannulation access, the typical circular cross-section of the tubular member 14 may be compressed and constrained in an oval or elliptical shape. By increasing the major axis of the oval, a larger area becomes available to target and penetrate by the user (e.g., a clinician, phlebotomist or patient). By increasing the major axis, the minor axis is decreased. Because the minor axis is decreased, the distance into which the needle 1000 may be inserted and still remain within the lumen of the tubular member 14 is decreased. The addition of the relatively puncture resistant region (e.g., the support member 12) opposite the cannulation zone(s) limits the occurrence of the user from passing through the back side of the tubular member 14. The inclusion of ovalizing (to increase cannulation zone) and the inclusion of a back wall zone resistant to puncture, combines to provide an improved graft construct.

In some embodiments, the support member 12 includes an at least partially radiopaque material such that the support member 12 may be viewed during delivery, deployment, and use. For example, the support member 12 may be viewed during radiography such that the user attempting to gain arterial or venous access may view the position of the support member 12 and thus gain access successfully and with fewer punctures. Various radiopaque markers may be positioned about the support member 12 and/or tubular member 14 to aid in delivery and implantation of the device 10.

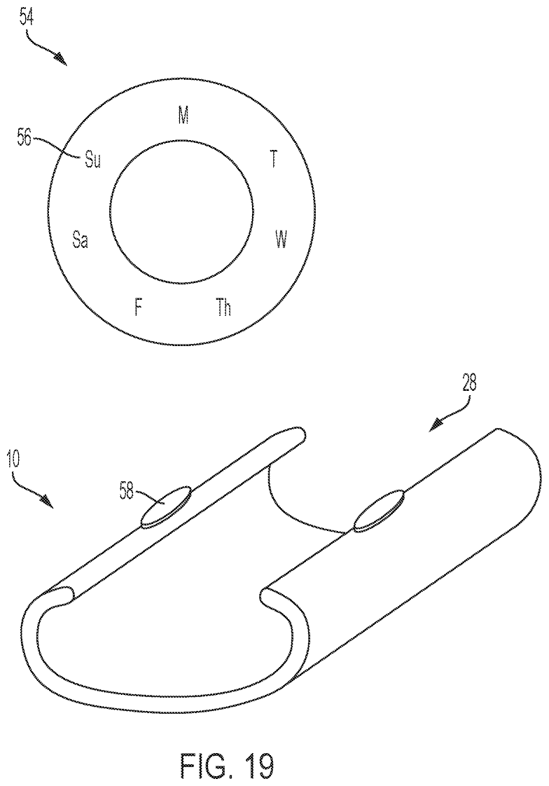

Referring to FIG. 19, in some embodiments, a location device 54 may be implemented in combination with the device 10 in order to aid locating, aligning and/or holding the device 10 steady. In one embodiment, the device 10 includes a portion 58 (e.g., adjacent the circumferential opening 28 of the support member 12) with magnetic properties (e.g., magnetized) or is attracted to a magnet (e.g., ferromagnetic). The location device 54 may include donut-shaped magnetic or ferromagnetic on the outside of the patient (e.g., the arm for dialysis) adjacent the device to magnetically attract (e.g., magnetically couple) together the location device 54 and the device 10. The device 10 is located, aligned, and/or held steady as the user (e.g., professional or patient for at-home dialysis treatment) locates the “target” to insert the cannulation needle 1000 through the donut hole of the location device 54. In some embodiments, the donut hole of the location device 54 may have indicators 56 (e.g., numbered/lettered marks) around the donut hole (e.g., similar to an analogue clock). The indicators 56 may be provided for each day so the user can find a different indicator for each day (e.g., a Tuesday mark) and insert the needle 1000 next to that indicator 56 and not reinsert at the same spot day after day.

In some embodiments, the support member 12 may be formed of a bioabsorbable or bioresorbable material. This allows the support member to be implemented in the body of a user for a limited time without having to perform a second operation to remove the support member 12. For example, regular IV access may be necessary for a limited time. The support member 12 may be implemented over that limited time and may slowly be resorbed by the patient, the resorption period being tuned to ensure that the support member 12 remains structurally sound during the expected period of use.

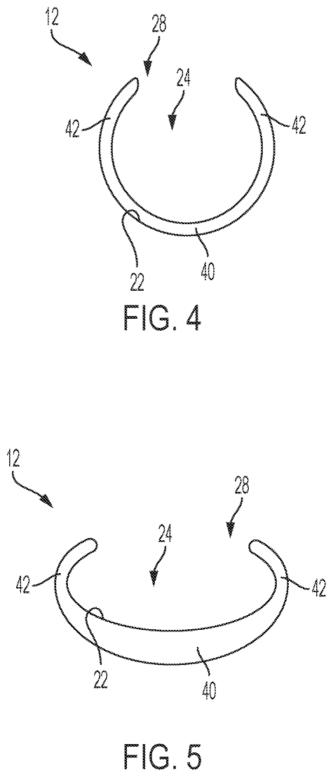

Referring to FIGS. 2 and 3, the interior surface 22 of the support member 12 presents a substantially oval cross-section. In other embodiments, such as that illustrated in FIG. 4, the interior surface 22 of the support member 12 presents a circular cross-section. It is understood that various other cross-sections may be implemented for various purposes. The interior surface 22 of the support member 12 having the discussed cross-sections is operable to retain the same cross-sectional area of the tubular member 14 within the receiving cavity 24 and externally of the receiving cavity 24. Stated otherwise, the interior surface 22 of the support member 12 determines the configuration in which the tubular member 14 will be maintained when engaged with the support member 12. The various configurations may provide various functionalities.

For example, referring to FIG. 1, the support member 12 includes an interior surface 22 with an oval cross-section, thus placing the tubular member 14 in an ovalized shape where the tubular member 14 is engaged with the support member 12. The ovalized shape of the tubular member 14 presents a relatively flat surface (as compared to a circular cross-section) along the length of the support member at the circumferential opening 28 of the support member 12 because the circumferential opening 28 is defined along the elongated portion of an oval. The relatively flat surface of the tubular member 14 provides an access site for the tubular member 14 that is easier to implement (e.g., for IV access including dialysis). The relatively flat profile of the tubular member 14 reduces the occurrence of an access needle glancing off a side of the tubular member 14 (i.e., the curved surface of the tubular member 14 is more difficult to penetrate unless the needle is aligned radially with circular profile). The flattened side wall of the tubular member 14 makes access reliable and easily obtained. In other embodiments, a circular profile may be desired in order to support the tubular member 14 in a circular profile configuration. For example, a native vessel may need to be supported in a circular profile (see FIG. 4). In some embodiments, by maintaining a circular profile of the tubular member 14 may provide appropriate fluid dynamics and degradation resistance. For example, in some applications such as dialysis, the tubular member 14 is positioned at a high pressure, high velocity position for blood flow, and the hemodynamics may be such that the circular profile of the tubular member 14 provides decreased or no disruption to the hemodynamics of the vessel. The circular profile of the tubular member 14 may also provide for greater longevity of the tubular member 14 during use.

Referring to FIGS. 2-5, the support member 12 is described in further detail. The support member 12 includes a body portion 40 and arm portions 42. The arm portions 42 extend from the body portion 40. The arm portions 42 each extend from opposite portions of the body portion 40 such that each of the arm portions 42 are opposing each other. The arm portions 42 define the circumferential opening 28 such that access to the receiving cavity 24 is provided between the arm portions 42 that oppose each other. The arm portions 42 are curved such that the arm portions 42 and the body portion 40 defines the receiving cavity 24. In some embodiments, the body portion 40 may also be curved (see FIGS. 2 and 3) and in some embodiments the body portion 40 is substantially flat (see FIG. 6). In some embodiments, the arm portions 42 comprise a material having a lower durometer relative to the durometer of the body portion 40. This allows the arm portions 42 to include the functionality of deflection to facilitate the tubular member 14 being inserted into the receiving cavity 24. Once the tubular member 14 is positioned in the receiving cavity 24, the arm portions 42, if deflected during insertion of the tubular member 14, return toward the undeflected configuration. The arm portions 42 may impart a compressive force to the tubular member 14 (e.g., for compression previously discussed). In some embodiments, at least the arm portions 42 are formed from a shape memory material such as nitinol. By including a shape memory material, the arm portions 42 may be able to impart increased compressive force. In some embodiments, the entire support member 12 is formed of a shape memory material and imparts compressive force to the tubular member 14 across a majority of the circumference of the tubular member 14.

Referring to FIG. 5, in some embodiments the body portion 40 is thicker than the arm portions 42. This may allow more flexion of the arm portions 42 while allowing the body portion 40 to retain the shape of the support member 12 more securely. The relatively thicker body portion 40 also allows the body portion 40 to have enhanced durability as a needle backstop. In some embodiments, the flexion of the arm portions 42 may facilitate bending in multiple bending planes.

It is understood that the support member 12 may further support the tubular member 14 to reduce twisting of the tubular member 14 during implantation. Twisting of the tubular member 14 during implantation can result in undesirable hemodynamics and may also result in kinking of the tubular member 14. By supporting the tubular member 14 with the support member 12, the tubular member 14 is held such as to reduce the risk of twisting of the tubular member 14.

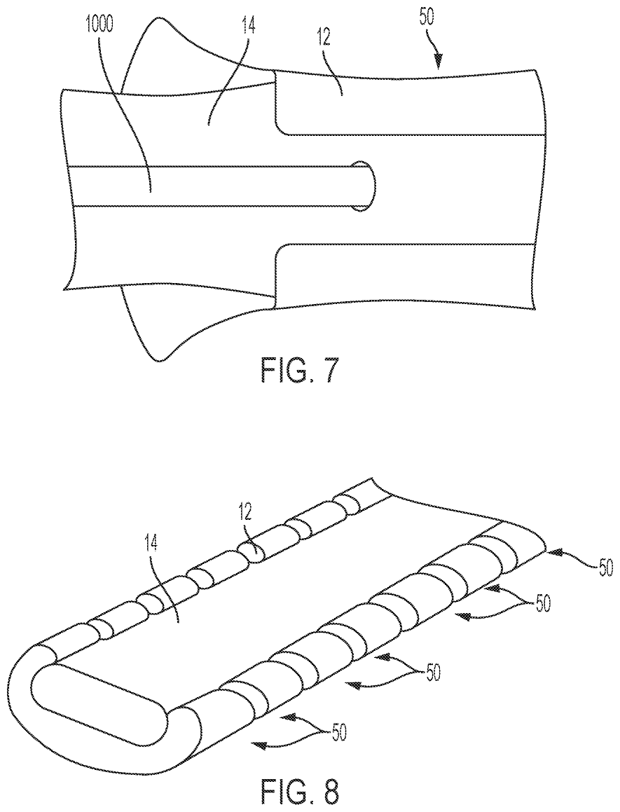

Referring to FIGS. 7-10, in some embodiments the support member 12 is segmented to include a plurality of longitudinal segments 50. Each of the longitudinal segments 50 is operable to at least partially surround a portion of the tubular member 14. The longitudinal segments 50 are operable to facilitate flexion of the elongate tubular member 14 when positioned with the support member 12. When installed on the tubular member 14, each of the longitudinal segments 50 are spaced from each other. By including a plurality of longitudinal segments 50 and having the longitudinal segments 50 spaced from each other, the tubular member 14 is operable to flex at least partially along the length where it is engaged with the support member 12. This allows the support member 12 to be implanted in the anatomy of a patient at a position where a bend or a curve is located. FIG. 8 depicts a tubular member 14 with a support member 12 coupled thereto, the support member 12 being segmented and the tubular member 14 being positioned in a bent configuration. Because the various longitudinal segments 50 are spaced from each other, the tubular member 14 can be manipulated to bend even when the support member 12 is substantially rigid. In some embodiments, as described above, the support member 12 may include a shape memory material (e.g., nitinol) that imparts flexibility to the support member 12 while also allowing the support member 12 to be resistant to puncture (e.g., rigid in at least one plane to be resistance to puncture by a needle 1000). The flexion of the support member 12 may be in a single bending plane or may be in multiple bending planes. As is seen in FIG. 8, the concave side of the curve of the bent tubular member 14 positions the arm portions 42 on the concave side in closer proximity to adjacent segments on the concave side than the arm portions 42 on the convex side are positioned to adjacent segments. The spacing of the various longitudinal segments 50 may be such that the space is slightly less than the outer diameter of a 12, 13, 14, 15, 16, 17, 18, 19, or 20 gauge needle. This limits the ability of the needle to travel through the space between the longitudinal segments.

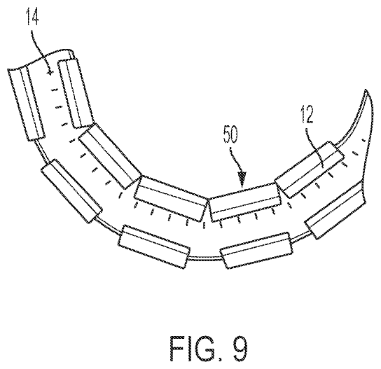

Referring to FIGS. 8 and 9, the longitudinal segments 50 may each be separate from each other (e.g., the longitudinal segments 50 are discrete elements). In this embodiment, each of the longitudinal segments may be installed onto the tubular member 14 separately. The longitudinal segments 50 may be spaced apart from each other any distance as determined by the user. The distance of each of the longitudinal segments 50 may be sufficient to allow the tubular member 14 to have a specific curvature imparted with the support member 12 installed on the tubular member 14. When the tubular member 14 includes or will be implanted with a greater curvature, the longitudinal segments 50 may be spaced from each other with a greater distance than when a lesser curvature is desirable, the lesser curvature allowing the longitudinal segments 50 to be positioned more closely together. In some embodiments, when the curvature is included or imparted on the tubular member 14, portions of adjacent longitudinal segments 50 may be in contact with each other on the inside of the curve whereas the longitudinal segments 50 are spaced from each other on the outside of the curve.

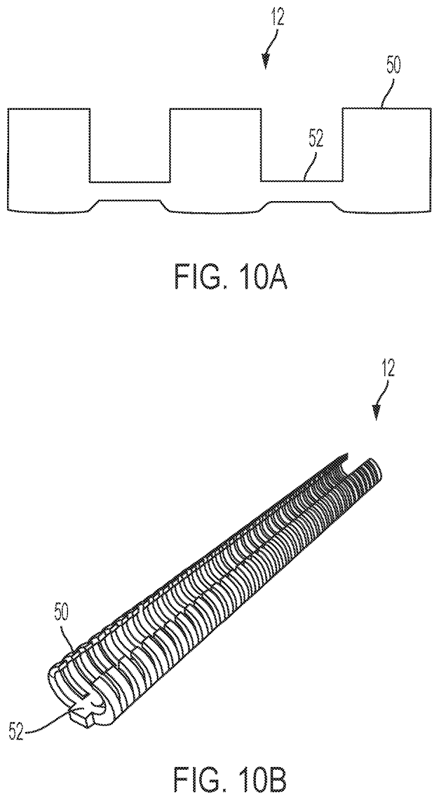

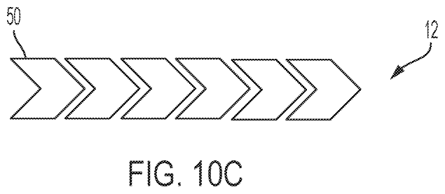

In other embodiments such as shown FIGS. 10A and 10B, the longitudinal segments 50 may be coupled to each other by an extension portion 52 (e.g., the plurality of longitudinal segments 50 are an integral member forming the support member 12, the support member 12 including an extension portion extending between adjacent longitudinal segments). The extension portion 52 may be integrally formed with each of the longitudinal segments 50, or the extension portion 52 may be coupled to the longitudinal segments 50 (e.g., welded to, adhered to, operable to receive, and so forth). The extension portion 52 positions the longitudinal segments 50 at fixed distanced from each other when in a neutral orientation. The extension portion 52 is operable to bend or deflect to facilitate a curvature of the tubular member 14. In some embodiments, the bending or deflection of the extension portion 52 is along a single plane of bending (e.g., bending along the extension portion 52). The extension portion 52 may be formed from a variety of materials including materials capable of both plastic and elastic deformation. For example, the extension portion 52 may be formed of nitinol to facilitate bending of the support member 12. The use of nitinol or other sufficiently resilient and/or rigid material may also allow the extension portion 52 to be resistant to puncture (e.g., puncture by a needle 1000). In some embodiments, the material thickness of the extension portion 52 is less thick than the thickness of the longitudinal segments 50, thus allowing the support member 12 to bend or deflect at extension portion 52 (e.g., the extension portion 52 includes a thinned profile operable to facilitate flexion). As illustrated in FIGS. 10A and 10B, the extension portion 52 may extend from the body portion 40 of adjacent longitudinal segments, thus leaving the arm portions 42 free from the extension portion 52. In some embodiments, the support member 12 is operable to place the tubular member 14 into a state of longitudinal compression. In some embodiments, the support member 12 may fabricated by densifying an expanded material such as ePE or ePTFE, 3-D printed (e.g., PE), or machined, cast, or molded.

Various configurations of the longitudinal segments 50 may be provided, including as illustrated in FIG. 10C. The various configurations may be implemented to accommodate various functionalities including bending profiles such as the radius of the curve or the curving in a single plane). For example, FIG. 10B demonstrates longitudinal segments 50 that include a nesting pattern in which the longitudinal segments 50 the shape of a chevron. An extension portion may or may not be implemented to connect each of the longitudinal segments 50 having this configuration. In some embodiments, the support member 12 may include differential gap widths between longitudinal segments 50. This may be along the length of the body of the support member 12 or may be on a lateral side as compared to the other lateral side (e.g., to facilitate one-way bending).

A method of assembling the support member 12 onto the tubular member 14 comprises elongating the tubular member so as to neck down an outer diameter of the tubular member 14 to be received within the receiving cavity 24 of the support member 12 (e.g., the C-shaped clip), wherein the receiving cavity 24 of the support member 12 has a smaller diameter than the outer diameter of the tubular member 14 when the tubular member 14 is not in a state of compression. In some embodiments, the outer dimension of the tubular member 14 when in the state of compression is in a range from 0.1% to about 10% relative to the outer dimension of the elongate tubular member when not in a state of compression. In some embodiments, the method includes deflecting the arm portion 42 away from the body portion 40 enlarging the circumferential opening 28 operable for the tubular member 14 to be received in the receiving cavity 24.

In some embodiments, the tubular member 14 may be the native vessel (e.g., an artery or vein of a patient) and the support member is placed about the native vessel to support the native vessel. In some embodiments, the tubular member 14 is formed of biological materials (e.g., an autograft, cadaver graft, and such). In some embodiments, the tubular member 14 is formed of synthetic material. Any of the materials used may be material that is operable to seal after puncture by a needle. The recovery and resealing of the tubular member 14 may be enhanced by the support member 12 holding the tubular member 14 in the state of compression in order to recover from a needle puncture (e.g., a 12-gage needle).

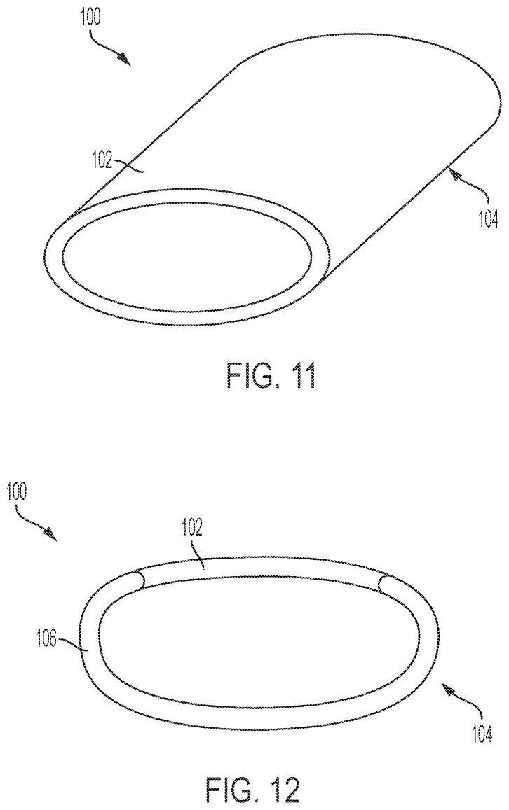

Referring to FIG. 11, in some embodiments, an AV access graft 100 is provided in which the AV access graft 100 includes an elastomeric, elongate tubular member 102 including a first longitudinal portion 104 having an oval-shaped cross section and resists deformation along the first longitudinal portion 104 such that the oval-shaped cross section at the first longitudinal portion 104 is retained when exposed to biological forces when implanted in a body. In some embodiments, the tubular member 102 is treated to include the oval-shaped cross section at a first longitudinal portion 104. In some embodiments, the elongate tubular member 102 is heat set to include the oval-shaped cross section at the first longitudinal portion 104.

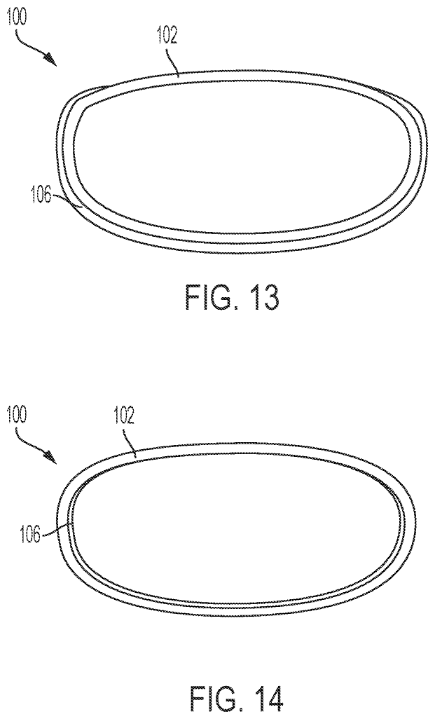

Referring to FIG. 12, in some embodiments, the tubular member 102 includes an integral support member 106 at the first longitudinal portion 104. In some embodiments, the tubular member 102 is treated to include the oval-shaped cross section. In some embodiments, the elongate tubular member 102 is heat set to include the oval-shaped cross section at the first longitudinal portion 104. In some embodiments, the integral support member 106 is a rigidified layer. The rigidified layer may be operable to provide support to retain a predetermined shape as well as it may be operable to provide a stop to resist needle puncture and/or provide tactile feedback from when a needle is positioned inside of the lumen of the elongate tubular member 102. The rigidified layer includes a sufficient durometer to prevent needle puncture through a backside of the tubular member 102 (e.g., Shore D or a higher durometer with a maximum value of about Mohs 10). In some embodiments, the integral support member 106 is a layer that is thermally adhered, chemically adhered, bonded, or otherwise attached to the tubular member 102. The layer may be on an exterior surface (see FIG. 13), an interior surface (see FIG. 14), or can be positioned at an entire thickness of the tubular member 102 along a portion of a circumference (see FIG. 12) of the tubular member 102.

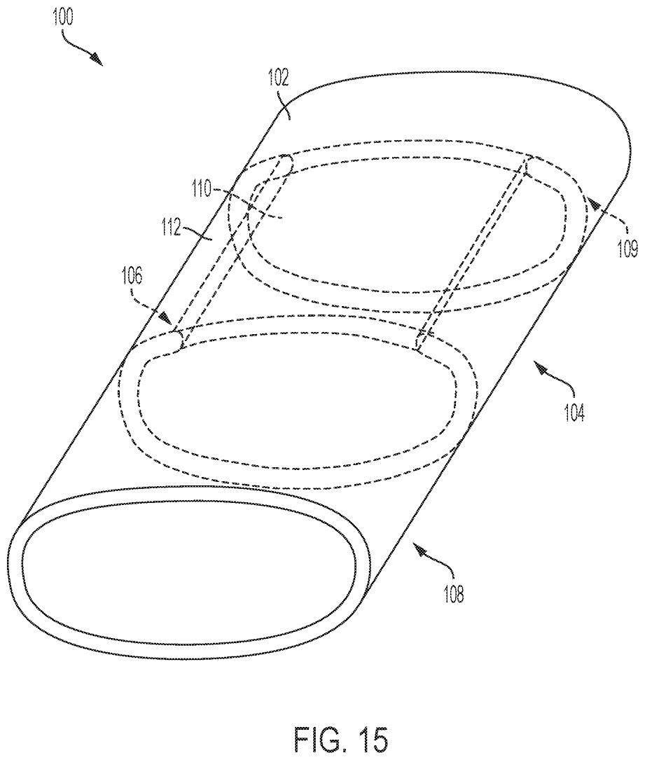

FIG. 15 illustrates the tubular member 102 including an integral support member 106 at the first longitudinal portion 104 (e.g., the tubular member 102 may include a supported portion for needle access and a non-supported portion) and the integral support member 106 not being present in at least second and third longitudinal portions 108, 109 of the tubular member 102. In some embodiments, the first longitudinal portion 104 at which the integral support member 106 supports the tubular member 102 may include a majority of the length of the tubular member. For example, the integral support member 106 may include about 40 cm of a 45 cm tubular member. However, the integral support member 106 may be provided along any portion or all of the length of the tubular member 102. In some embodiment, the tubular member 102 is free from the support member 106 at each end of the tubular member 102. This allows a surgeon to cut the tubular member 102 to a desired length and angle without having to cut through the support member 12. With respect to scenarios in which the device 10 is implemented for cannulation, support members 12 may be implemented in the cannulation zones, but may be considered optional in the other areas.

The support member 106 may at least partially impart the oval-shaped cross section. In some embodiments, the tubular member 102 at the first longitudinal portion 104 includes a first elongated portion 110 and a second elongated portion 112 defining the oval-cross section, wherein the first elongated portion is operable to self-seal after a puncture. In some embodiments, the second elongated portion 112 is reinforced to resist puncture. It is understood that various other cross-sectional profiles may be implemented. For example, a circular cross section may be implemented, the circular cross section being at least partially imparted and maintained by the integral support member 106. As illustrated in FIG. 15, the support member 106 is positioned around approximately 75% or 270 degrees of the tubular member 102. It is understood that the support member 106 can be positioned about various portions of the tubular member 102 in various embodiments. For example, the support member 102 may be positioned about the support member over 180-350 degrees of the tubular member 102 (e.g., from about 180 degrees to about 225 degrees, from about 225 degrees to about 270 degrees, from about 270 degrees to about 315 degrees, and from about 315 degrees to about 350 degrees). By providing greater regions of support on the tubular member 102, there is less likelihood of a needle passing through the backside of the tubular member 102 during puncture. With the ovalized configuration, the unsupported region may be more easily accessible as previously discussed.

The device 10 may transition from substantially circular to substantially oval along the length of the device 10 at multiple longitudinal positions. For example, the tubular member 102 may be round at one end, then gradually transition to a first oval, then transition back to round, then gradually transition to second oval, then transition back to round at the other end. The two ovalized areas are cannulation zones (for example, in the case of AV Access). A user is able to feel these areas (through the skin) to know where the ovalized sections of the device 10 are. In some embodiments, as the device 10 transitions between various cross-sectional shapes, the inner cross-sectional area of the device 10 (e.g., the cross-section area of the lumen) remains constant. This limits flow disruptions and thrombosis at the areas of transition.

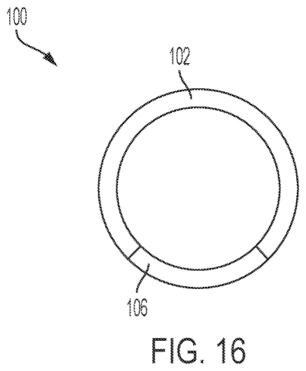

Referring to FIG. 16, another embodiment of the tubular member 102 is provided in which the support member 106 is positioned around approximately less than 50% percent of the circumference of the tubular member 102. A circular profile of the tubular member 102 is illustrated, however it is understood that any number of configurations may implement the support member 106 being positioned around approximately less than 50% percent of the circumference of the tubular member 102. It is also understood that the various configurations discussed above with the integral support member 106 being positioned on an interior surface, exterior surface, or including the entire thickness of the tubular member at a portion of the circumference of the tubular member 102 may be implemented with respect to a support member 106 being positioned around approximately less than 50% percent of the circumference of the tubular member 102. For example, the support member 102 may be positioned about the support member over 10-180 degrees of the tubular member 102 (e.g., from about 10 degrees to about 45 degrees, from about 45 degrees to about 90 degrees, from about 90 degrees to about 135 degrees, and from about 135 degrees to about 180 degrees). By providing a support member over less than 50% of the circumference of the tubular member 102, the surface area is increased for accessing the lumen of the tubular member 102 while still providing a backstop to reduce needle puncture through the backside of the tubular member 102.

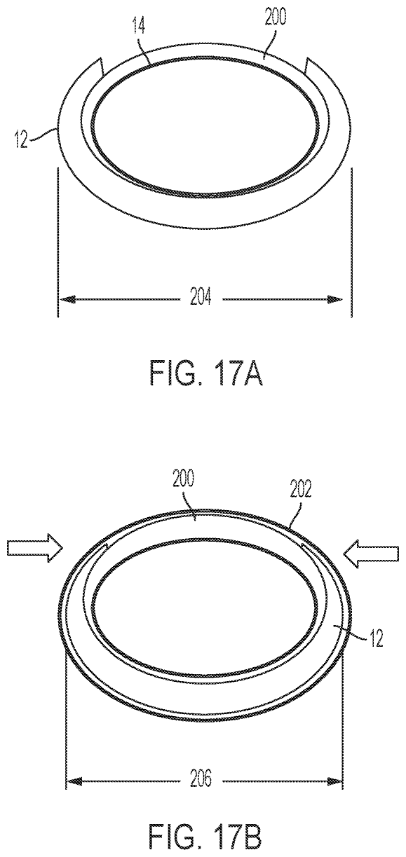

Referring to FIGS. 17A and 17B, a device 10 is shown including a tubular member according to any of the embodiments discussed herein (e.g., tubular member 14 or tubular member 102) and a support member according to any of the embodiments discussed herein (e.g., support member 12 or integral support member 106). The support member 12 and the tubular member 14 are coupled to each other via an adhesive 200. An overwrap 202 may be applied over the assembly. The overwrap 202 may impart compressive force (shown by arrows in FIG. 17B) to the tubular member (and in some embodiments, increased compressive force in addition to the compressive force imparted by the support member). Prior to application of the overwrap 202, the device 12 may include a first diameter 204 along the major axis. After the overwrap 202 has been applied, the device 12 may include a second diameter 206 along the major axis where the second diameter 206 is less than the first diameter 204. This occurs because of the compressive force applied by the overwrap 202.

As previously discussed, the embodiments discussed herein may be implemented in a variety of contexts are not limited to any specific embodiment discussed herein. For example, the use of the device 10 for cannulation (e.g., in hemodialysis) is discussed at length. However, other applications are applicable such as use in the side of anastomosis and therefore the descriptions provided herein are not limited to cannulation. For example, increased durometer of the device may be implemented at the site of anastomosis, where the increased durometer mitigates suture-hole bleeding. Another example may include when the support member 12 is implemented on native vessels. For example, the support member 12 may be implemented at positions at which blood vessels are compressed by other anatomy competing for the same space within the body. This occurs in, for example, May-Thurner Syndrome, in which the left iliac vein is compressed by the right iliac artery. Compression of the left iliac vein has the potential to cause deep vein thrombosis. The support member 12 may be implanted externally to a vessel (e.g., the left iliac vein) to restore blood flow and/or competence to the vessel.

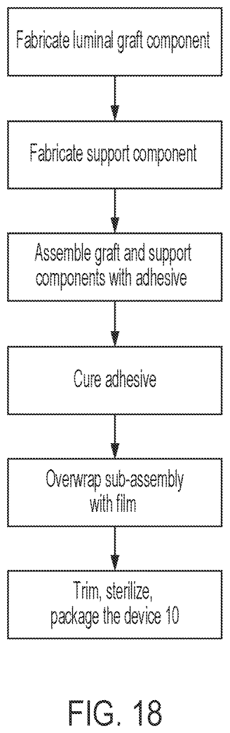

Referring to FIG. 18, a method of manufacturing a device 10 is provided. The method includes fabricating a luminal graft component (e.g., an elongate tubular member 14, fabricating a support component (e.g., a support member 12), assembling the graft and support components with an adhesive (e.g., any suitable elastomeric adhesive), curing the adhesive, overwrapping the sub-assembly with a film (e.g., a film appropriate for abluminal use), and trimming, sterilizing, and packaging the device.

In any of the embodiments discussed herein, a tubular member 102 may include a variety of materials and compositions, including biocompatible materials operable to self-seal after puncture. Such materials may be implemented, for example, when intravenous access is needed soon after the tubular member 102 is implanted. Other biocompatible materials may be implemented that are operable to facilitate tissue ingrowth allowing the tissue to provide sealing capabilities after sufficient tissue has been incorporated into the material. Materials may be selected for their various properties such as durability, durometer, thickness, and so forth to optimize the tubular member for its applications (e.g., for high velocity flow, life of the implant, low profile of the implant, and so forth). In some embodiment, the tubular member 102 is formed of a single layer of material.

A biocompatible material for the tubular member components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, a polyethylene terephthalate, fluorinated ethylene propylene, polyethylene (PE), expanded polyethylene (ePE), or another biocompatible polymer or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers. Biological grafts may also be implemented, including, but not limited to, a bovine carotid artery or a lab-grown vessel.

In some embodiments, it is understood that the support member may include a surface texture to either promote tissue ingrowth or to exclude tissue incorporation. This may be accomplished in various ways, including, but not limited to, providing a smooth surface, a textured surface, a porous surface, a treated surface, and so forth.

In some embodiment the tubular member and support member of any of the embodiments discussed herein may be provided with a straight configuration or a curved configuration. The curved configuration may provide a stress-free state when implanted when the device is positioned with at least one curve (e.g., as an arteriovenous graft).

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A device for supporting an elongate tubular member, the device comprising:
a support member having an interior surface defining a receiving cavity, the support member further defining a circumferential opening, wherein the receiving cavity is operable to receive an elongate tubular member therein, the support member operable to place the elongate tubular member into a state of compression about the circumference.

2. The device of claim 1, wherein the receiving cavity is adhesively coupled to the elongate tubular member.

3. The device of claim 1, wherein the support member comprises a material resistant to puncture by a needle at typical forces exerted manually by a user.

4. The device of claim 3, where the support member comprises a bioabsorbable material.

5. The device of claim 1, wherein the circumferential opening of the support member is from about 10% to about 90% of a circumference of elongate tubular member.

6. The device of claim 1, wherein the interior surface of the support member presents an oval cross-section.

7. The device of claim 1, wherein the interior surface of the support member presents a circular cross-section.

8. The device of claim 1, wherein the support member is operable to retain the same cross-sectional area of the elongate tubular member within the receiving cavity and external of the receiving cavity.

9. The device of claim 1, wherein the support member has two opposing arms defining the circumferential opening, the two opposing arms being interconnected by a body and including a material having a durometer that is lower than a durometer of the body.

10. The device of claim 8, wherein the two opposing arms impart a compressive force to the elongate tubular member.

11. The device of claim 9, wherein the two opposing arms portions are formed from a shape memory material.

12. The device of claim 11, wherein the shape memory material is nitinol.

13. The device of claim 9, wherein the body is thicker than the two opposing arms.

14. The device of claim 1, wherein the support member includes a plurality of segments along a longitudinal length operable to facilitate flexion of the elongate tubular member when positioned with the support member.

15. The device of claim 14, wherein the plurality of segments are discrete elements.

16. The device of claim 14, wherein the plurality of segments are an integral member forming the support member, the support member including an extension portion extending between adjacent segments, the extension portion including a thinned profile operable to facilitate flexion.

17. The device of claim 14, wherein the plurality of segments facilitates flexion of the elongate tubular member.

18. The device of claim 14, wherein the support member is resistant to puncture.

19. The device of claim 1, wherein the support member is operable to place the tubular member into the state of compression.

20. The device of claim 1, further comprising a location device operable to magnetically coupled to the support member, the location device forming a donut hole through which a needle may be inserted.

21. The device of claim 15, wherein the location device includes indicators about a periphery of the donut hole indicating different positions for needle insertion.

22.-25. (canceled)

26. An AV access graft, comprising:
an elongate tubular member defining a lumen; and
an elongate support member defining a receiving cavity and defining a circumferential opening, wherein the receiving cavity is operable to receive the elongate tubular member therein, the elongate support member operable to place the tubular member into a state of circumferential compression.

27.-33. (canceled)

34. An AV access graft comprising:
an elastomeric, elongate tubular member including a first longitudinal portion having an oval-shaped cross section and resists deformation along the first longitudinal portion such that the oval-shaped cross section at the first longitudinal portion is retained when exposed to biological forces when implanted in a body.

35.-41. (canceled)

42. An AV access graft comprising:
an elongate tubular member including a first longitudinal portion having an integral support member over a portion of a circumference of the elastomeric, elongate tubular member at the first longitudinal portion.

43.-55. (canceled)

56. A device for supporting a tubular member, the device comprising:
a segmented support member including a plurality of longitudinal segments, the segmented support member having an interior surface defining a receiving cavity, wherein the receiving cavity is operable engage the tubular member therein, each longitudinal segment in the plurality of longitudinal segments at least partially surrounding a portion of the tubular member.

57.-71. (canceled)
